(19) United States
(12) Patent Application Publication
(10) Pub. No.: US 2004/0148667 A1
(43) Pub. Date: Jul. 29, 2004

(54) Means for identifying nucleotide sequences involved in apomixis

(75) Inventors: Yves Savidan, Clarensac (FR); Daniel Grimanelli, Mexico (MX); Enrico Perotti, Mexico (MX); Olivier Leblanc, Mexico (MX)

Correspondence Address: FOLEY AND LARDNER, SUITE 500, 3000 K STREET NW, WASHINGTON, DC, 20007, US

(73) Assignee: Institut De Recherche Pour Le Developpement (IRD)

(21) Appl. No.: 10/668,322

(22) Filed: Sep. 24, 2003

Related U.S. Application Data
(63) Continuation of application No. 09/375,415, filed on Aug. 17, 1999.
Application No. 09/375,415 is a 371 of international application No. PCT/FR98/00308, filed on Feb. 17, 1998.

(30) Foreign Application Priority Data
Feb. 17, 1997 (FR) 97/01821

(51) Int. Cl. C12Q 1/68; A01H 5/00

(52) U.S. Cl. 800/320.1; 435/6

(57) ABSTRACT

The invention concerns a method for identifying in Gramineae, more particularly in maize, a nucleotide sequence involved in the apomixis in apomictic plants. The method relates to identifying the genome of the Gramineae, by phenotypic analysis, genetic mapping and marking by means of transposons, of the meiotic mutations whereof the corresponding gene is shown to be orthologous to genes involved in the expression of apomixis. The invention also concerns the use of a cloned gene in the Gramineae to identify and isolate the orthologous gene sequence in apomictic plants. The invention further concerns the use or modification of the isolated sequence in apomictic forms for inducing an apomictic development in sexual plants.

The invention relates to means for identifying, isolating and characterizing nucleotide sequences involved in apomixis.

It more particularly relates to a process and tools for identifying these sequences in the genome of apomictic plants, and then for isolating them and characterizing them.

It also relates to transgenic applications implemented with the aid of these sequences, and to the products obtained.

In its modern meaning, apomixis, or agamospermy, summarizes all the phenomena of asexual reproduction by seed. Apomictic plants are found in almost 300 species of angiosperms belonging to more than 35 families. The various forms of apomixis, which generally only affect female reproduction, are characterized by the absence of meiotic reduction, the absence of fertilization of the oosphere, and parthenogenetic development of embryos. Apomixis thus leads to the production of descendants genetically identical to their parent plant.

The natural opposite of apomixis is sexual reproduction, or amphimixis. In contrast to apomixis, sexual reproduction includes processes comprising both reductional meiosis and syngamy.

Meiosis distributes the homologous chromosomes resulting from the parents randomly between the gametes. It also allows recombination between homologous chromosomes, by the intermediary of crossing-over. Syngamy is the fusion of the gametes. It allows a particular combination of the genetic information resulting from the two parents to be joined together in an individual. Amphimixis thus produces genetically unique descendants by recombination of parental genomes.

In the development cycles of plants, alternation of two successive generations separated by meiosis and fertilization are thus observed. The first generation corresponds to the sporophyte. One or more cells of the sporophyte undergoes meiosis, producing meiospores. The meiospores develop into gametophytes, which represent the gametophytic generation at the origin of gametes. The fusion of gametes leads to the zygote, which represents the return to the sporophytic generation.

In sexual angiosperms, the female gametophyte (the embryonal sac) develops into a multicellular structure which is very different from the sporophyte, the ovule. In the course of development of the ovule, a particular cell, the archesporial cell, passes through two successive stages: megasporogenesis (formation of a reduced megaspore, or meiospore, from an archesporial cell) and megagametogenesis (formation of the female gametophyte from a megaspore) to produce a pluricellular gametophyte which contains a single gamete, the oosphere. This type of development (polygonum type) involves almost 80% of angiosperms. The various forms of apomixis correspond to a series of variations of this theme.

The origin of the embryo allows a first subdivision between two fundamental forms of apomixis. In cases of adventitious embryony, the embryos differentiate directly from somatic cells of the nucellus or of the tegument of the ovule. There is therefore no alternations of sporophytic and gametophytic generations. Gametophytic apomixis, on the other hand, is characterized by the formation of a non-reduced female gametophyte, and parthenogenetic development of the embryo from the oosphere. In the text which follows, any reference to apomixis will relate to gametophytic apomixis.

Two major types of gametophytic apomixis are observed, which differ in the origin of the female gametophyte. In aposporic forms, the non-reduced embryonal sac is derived from a somatic cell of the ovule, generally of the nucellus. In diplosporic forms, it results from a generative cell, the archesporial cell. Apomeiosis covers both apospory an diplospory. There is in fact a wide diversity of processes leading to the formation of a non-reduced gametophyte.

In sexual angiosperms, the male gametophyte (the grain of pollen) contains two spermatozoids. One fertilizes the oosphere, giving birth to the embryo, while the other combines with the nucleus of the central cell, at the origin of the albumen. The embryo and the albumen are thus both sexed. Double fertilization is referred to, a characteristic inherent to angiosperms. In the majority of apomictic plants, the embryo develops without fertilization, but the albumen becomes sexed. Pseudogamy, or pseudogamic apomixis, is referred to when the fertilization of the central cell is necessary for the development of the albumen, and autonomous apomixis is referred to when both the embryo and the albumen develop without fertilization.

At the embryonal level, apomixis thus actually corresponds to asexual reproduction by seed. It results from a sum of clearly identifiable components: apomeiosis, or formation of an embryonal sac without meiotic reduction, and parthenogenesis, or formation of an embryo without fertilization of the oosphere. Apomeiosis and parthenogenesis ensure alternation of sporophytic and gametophytic generations, but without alternation of the nuclear phases: the sporophyte and gametophyte maintain the same level of ploidy.

In a plant, however, apomixis is in fact a mode of mixed reproduction, combining amphimixis and asexual reproduction. In effect, as a general rule apomixis is a facultative phenomenon: it appears in the descendants of apomictic plants of “out-of-type” individuals, that is to say those which are genetically different from their parent plant. For apomictic development in the strict sense, it is necessary for the two conditions of non-reduction and non-fertilization to be combined. Out-of-type individuals can appear if one or both conditions are not met. Depending on the realization or failure of meiosis and fertilization, the possible classes of descendants in a facultative apomictic plant are the following:

| | Fertilization | Non-fertilization |
|---|---|---|
| Meiosis | n + n | n + 0 |
| Apomeiosis | 2n + n | 2n + 0 |

In the definition of the hybrids of the type “n+n” or “2n+n” etc . . . , the first term designates the state of the gametophyte, reduced (n) or non-reduced (2n). The second term illustrates the presence or absence of fertilization of the oosphere. The category “2n+0” thus represents apomixis stricto sensu, and the category “n+n” represents amphimixis. The category “2n+n” leads to a genomic accumulation, and the category “n+0” leads to haploidization of the parent plant. The respective proportions of these different categories vary from one species to another, and even from one plant to another within a given species. In the description which follows, references to apomixis relate both to the facultative apomictic forms and to the obligatory apomictic forms producing exclusively descendants of the type 2n+0.

The genetic determinism of apomixis is still very poorly understood. It is now agreed that angiosperms with asexual reproduction descend from ancestors with sexual reproduction, and that the transition from one form of reproduction to another reveals a genetic determinism. Apomixis is thus said to result from expression of apomixis genes or alleles, that is to say those present and expressed in apomictic plants but absent or non-functional in sexed plants.

The large majority of works on genetic control of apomixis relate to apomeiosis, and more frequently apospory. There is currently quite broad consensus on the principle of a Mendelian heredity of apospory (see references (1) and (2), the bibliographic references being given at the end of the description). There is little knowledge of diplospory, but the results which exist (3, 4) show that the hypothesis agreed for aposporic plants without doubt applies to diplosporic plants. In these different models, the mode of action of the genes in question remains an enigma. These analyses tend to show, however, that the gene responsible for apomeiosis could in itself trigger the entire apomictic process.

Apomixis arouses great interest with regard to its potential for improving plants. The use of apomixis in the main crop plants would represent a simple way of fixing heterosis. It is potentially a revolution in manipulation of reproduction systems.

Several programmes have emerged, the aim of which is to characterize the “apomixis genes” and to introduce them into crop plants.

The oldest and probably the most advanced works use naturally apomictic plants. The corresponding alleles are present in them, and functional. Their transfer into important cultivated species can be achieved either by the intermediary of interspecific crossing between a cultivated species and an apomictic relative, or by isolating the corresponding genes, to then introduce them into the target species by transgenesis ((5); (6); (7) and (8)).

Another approach undergoing rapid development comprises generating apomixis in sexed species by various methods of mutagenesis. Arabidopsis thaliana is the model used the most ((9), (10) and (8)). Equivalent works are in progress in the petunia, Petunia hybrida (10), and Hieracium (8). In all the cases in question, the fundamental hypothesis is that it is a matter of simple genetic control, and that the transfer or modification of a very small number of alleles provides sufficient conditions for expression of apomixis. Apomixis here is understood as the result of the abovementioned events: failure of meiosis and parthenogenesis.

The works by the inventors in this field led to them to investigate in maize orthologous genes to that or those involved in the expression of apomixis in the species of the genus Tripsacum, a genus related to maize. In the description and the claims, “orthologous genes” is understood as meaning genes which would have diverged from a common gene, or paralogous genes, at the same time as the species which carry them. The intended genes have the same functions with respect to apomixis.

The genus Tripsacum belongs to the Andropogon tribe. It is the only known relative of the genus Zea on the American continent. (4) and (11) have carried out the most complete study of the modes of reproduction on Tripsacum. These works have enabled the following points to be made:

all the polyploidal accessions reproduce by diplosporic apomixis,

the non-reduction in the apomictic forms is chiefly of the Antennaria type, with rare occurrence of the Taraxacum type,

the embryonal sacs in the diplosporic forms result directly from megasporocytes by three successive mitoses,

the failure of meiosis in the diplosporic forms is associated with the absence of depots of callose around the megasporocytes,

analysis of an F1 population between maize and a diplosporic form of Tripsacum indicates a simple heredity of apomeiosis,

various alleles detected by molecular maize probes have been mapped close to the locus responsible for apomeiosis; these are probes umc28, csu68 and umc62,

these probes enable a partial homology relationship to be established between the chromosome responsible for apomeiosis in Tripsacum and the distal part of the long arm of chromosome 6 in maize.

The works of the inventors led them to propose and demonstrate that the genes responsible for apomixis in Tripsacum have one or more orthologous genes in the genome of sexed Gramineae, in particular in the genome of maize. This approach allowed the development of a general strategy leading to the identification and then cloning of the nucleotide sequences responsible for apomixis, and development of new tools for its implementation.

The object of the invention is thus to provide a process for identifying in a Gramineae, and more particularly in a maize, an orthologous gene to a gene involved in apomixis.

An object is also to provide a process for isolating the sequence of this gene.

An object of the invention is also the use of this sequence to isolate the corresponding sequences of orthologous genes in apomictic plants and the use of these sequences in transgenesis.

An object is also a process which allows confirmation of the functional relationship between the sequences isolated in the apomictic plants and phenotypical expression of apomixis.

The process according to the invention for identifying in a Gramineae, and more particularly in a maize, a nucleotide sequence orthologous to the sequence responsible for all or some of the apomictic development in an apomictic form is characterized in that mutations having a phenotypical expression close or similar to that observed in an apomictic form are mapped in the genome of the Gramineae, more particularly in that of a maize, in order to identify those mutations which appear orthologous to genes involved in apomixis.

A related phenotype is identified here on the basis of four characteristics of the diplosporic forms, which can be observed either independently or in conjunction: (a) the mutations are specific to megasporogenesis and do not affect the male reproductive function, (b) they lead to the formation of non-reduced gametes from an archesporial cell, (c) they are characterized by the absence of depots of callose around the parent cells of the megaspore, (d) the control points which usually act during the formation of the embryonal sac seem inactive, and the embryonal sacs are formed normally in spite of the failure of one stage in the course of megasporogenesis.

The reference in the process defined above to the identification of nucleotide sequences comprises identification of loci or genes in one embodiment of the process of the invention, or mapping in the genome of the Gramineae, more particularly that of a maize, of the meiotic mutations to identify those which seem orthologous to genes involved in apomixis.

The invention particularly relates to a process for identifying in a Gramineae, more particularly in a maize, an orthologous gene sequence to that of the gene which controls apomeiosis in the apomictic forms, characterized in that the phenotypical expression is studied and in that the position of various meiotic mutations in the genome of the Gramineae, more particularly of a maize, is located with the aid of molecular markers which are capable of locating the loci responsible for apomeiosis in the said apomictic form.

Generally, the mutations located according to the invention are cloned and sequenced.

The inventors have demonstrated in particular that the genes involved in the expression of apomixis in Tripsacum have one or more orthologous genes in the genome of maize.

The use according to the invention of the same set of markers in maize and Tripsacum has allowed identification of candidate genes having both (1) the same genomic location as the genes which control diplospory, in the sense that they are situated in the same chromosomal region of a segment which, in maize, is homeologous to that which controls diplospory in Tripsacum, and (2) a related phenotype, depending on the abovementioned criteria. Preferably, the location relates to the elongate and afd loci in the genome of maize.

The process of the invention is also characterized in that it comprises tagging the meiotic mutations located, with the aid of transposons. The invention particularly relates to tagging of the elongate locus with the aid of transposons.

The use of transposons of which the sequence is known allows creation of a mutation for a gene of which only the phenotypical expression is known. The insertion of the transposon into the gene which is to be isolated is often characterized by the loss of its function. In the case of recessive alleles, it is frequently characterized by the appearance of the recessive phenotype in heterozygous plants. Particularly interesting transposons comprise transposable elements of the Mutator or Ac/Ds type.

Cloning and sequencing of the mutations located are advantageously carried out.

A mutated gene can thus be isolated by marking the site of insertion of the transposon, the various loci where transposons have been inserted being cloned by the conventional techniques of Mendelian analysis and molecular biology (12) and (13), and sequenced if desired.

In the case, for example, of the site corresponding more particularly to the elongate locus, the site for expression of the allele el1 is first characterized phenotypically. It is then located by genetic mapping, and its position is compared with that of the loci which control diplospory in Tripsacum. The loci are then marked by the intermediary of transposons, isolated and then sequenced.

The invention also relates to the use of at least part of a sequence as defined above for identifying and then isolating the sequence or orthologous genes in apomictic forms.

The invention relates, as such, to the nucleotide sequences isolated. These sequences are characterized in that they are orthologous to sequences responsible for all or some of the development in an apomictic form. The invention also relates to sequences which are homologous, in terms of function, to those identified above.

The invention particularly relates to a nucleotide sequence of this type corresponding to the mutated elongate gene.

The invention particularly relates to a nucleotidic sequence containing a fragment homologous to histone H1-1 of Arabidopsis thaliana and to the yeast gene Cd36 as detailed in the examples. More particularly, the invention relates to a nucleotidic sequence containing or consisting of SEQ ID N° 1.

The invention also relates to the nucleic acids containing one or more sequences as defined above together with the regulatory sequences necessary for expression in the plant material.

The invention also relates to the cloning and expression vectors containing such nucleic acids and to the cell hosts containing these vectors, for example Agrobacterium tumefaciens.

The invention also relates to the use of such sequences, where appropriate in conjunction with other alleles characteristic of apomictic forms, for transforming the genome of a plant material, plant cells, plants in various stages of development, and seeds, in order to confer on them an apomictic development. The said alleles correspond to genes other than the orthologous genes provided by the invention.

The invention particularly relates to a process for producing apomictic plants, characterized in that a sequence of the mutated elongate gene as defined above is used.

This transformed plant material as such is included in the scope of the invention and is characterized by the fact that it contains in its genome at least part of the said sequence involved in an apomictic development, where appropriate in conjunction with other alleles characteristic of apomictic forms.

The cells, plants and seeds envisaged belong to the family of Gramineae. They are, in particular, from maize.

The transformation of the plant material, cells, plants and seeds is advantageously achieved by applying the conventional techniques of transgenesis.

By way of example, for obtaining transgenic maize plants.

A. Obtaining and use of the Callus of Maize as the Target for Genetic Transformation.

The genetic transformation of maize, regardless of the method employed (electroporation, Agrobacterium, microfibres, particle gun) generally requires the use of undifferentiated cells in rapid division which have preserved an aptitude for regeneration of whole plants. This type of cell makes up the friable embryogenic callus (so-called type II) of maize.

These calli are obtained from immature embryos of genotype H1 II or (A188×B73) by the method and on the media described by Armstrong (1994). The calli thus obtained are multiplied and maintained by successive sub-culture every fortnight on the initiation medium.

Plantlets are then regenerated from these calli by modifying the hormonal and osmotic equilibrium of cells by the method described by Vain et al. (1989). These plants are then acclimatized in a greenhouse, where they can be crossed or autofertilized.

B. Use of a Particle Gun for Genetic Transformation of Maize.

The above paragraph describes the obtaining and regeneration of cell lines necessary for the transformation; a method for genetic transformation leading to stable integration of modified genes into the genome of the plant is described here. This method relies on the use of a particle gun; the target cells are fragments of calli described in paragraph 1. These fragments of surface area 10 to 20 mm² have been positioned, 4 h before bombardment, in an amount of 16 fragments per dish, in the centre of a Petri dish containing a culture medium identical to the initiation medium, to which 0.2 M mannitol+0.2 M sorbitol have been added. The plasmids which carry the genes to be introduced are purified over a Qiagen column in accordance with the manufacturer's instructions. They are then precipitated on to tungsten particles (M10) in accordance with the protocol described by Klein et al., Nature, 1987, 327, pages 70-73. The particles coated in this way are projected towards the target cells with the aid of the gun and in accordance with the protocol described by J. Finer (1992).

The dishes of calli bombarded in this way are then sealed with the aid of Scellofrais®, and then cultured in the dark at 27° C. The first sub-culture takes place 24 h thereafter, and then every fortnight for 3 months on medium identical to the initiation medium, to which a selective agent has been added. The selective agents which can be used generally consist of active compounds of certain herbicides (Basta®, Round up®) or certain antibiotics (hygromycin, kanamycin etc).

After 3 months or sometimes sooner, calli of which the growth is not inhibited by the selection agent are obtained, usually and in the majority of cases composed of cells resulting from the division of a cell which has integrated into its genetic heritage one or more copies of the selection gene. The frequency in which such calli are obtained is about 0.8 callus per dish bombarded.

These calli are identified, individualized, amplified and then cultured in order to regenerate plantlets. To avoid any interference with non-transformed cells, all these operations are conducted under culture media containing the selective agent.

The plants regenerated in this way are acclimatized and then grown in a greenhouse, where they can be crossed or autofertilized.

C. Use of Agrobacterium tumefaciens for Genetic Transformation of Maize.

The technique used is that described by Ishida et al. (Nature Biotechnology, 1996, 14: 745-750) or by Horsch et al. Science, 1984, 223, pages 496-498.

The invention thus provides means for providing a population of apomictic plants which have active transposable elements.

In particular, it provides means for inducing apomictic development in a sexed plant, and in particular in maize.

In another application according to the invention, at least part of a sequence as defined above is used to identify and isolate the orthologous locus sequence in apomictic forms.

The invention thus relates to the hybridization probes compiled from the said sequences and to the primers which can be used in PCR techniques.

Such probes and primers correspond, in particular, to those compiled from the elongate sequence.

The hybridization or PCR techniques are advantageously carried out by conventional methods.

The invention also relates to a process for identifying and isolating a gene responsible for diplospory in apomictic Tripsacum, characterized in that at least part of the elongate locus sequence is used.

The process of the invention is also characterized in that the sequence isolated in apomictic Tripsacum is used for functional analysis of the relationship between this sequence and the expression of apomixis. A mutagenesis process, as illustrated in the examples, is used in particular, allowing confirmation of the relationship between the sequence isolated in apomictic Tripsacum from the elongate gene sequence and the phenotypical expression of apomixis.

According to the invention, the relationship between the said sequence and the expression of apomeiosis is confirmed in particular.

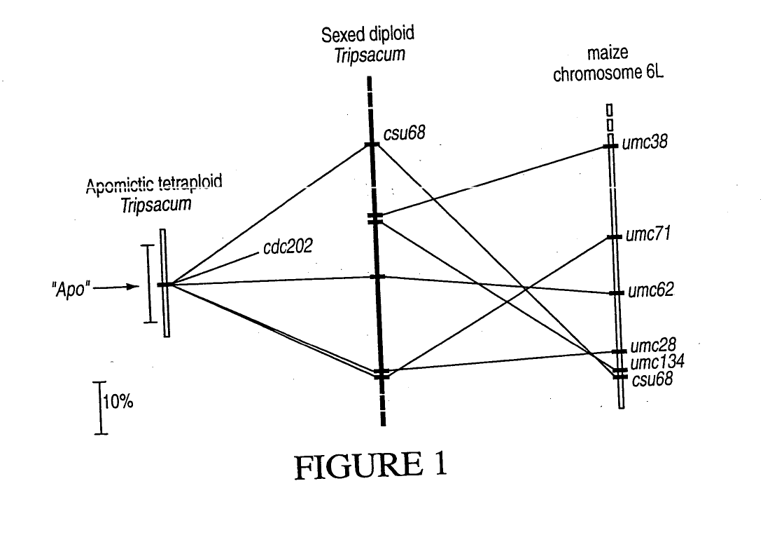

Other characteristics and advantages of the invention are given in the examples which follow. In these examples, reference is made to FIGS. 1 to 5, which show, respectively:

FIG. 1: Genetic mapping of the chromosomal segment which controls diplospory in an apomictic tetraploid Tripsacum, and the comparison with sexed diploid plants in Tripsacum and maize,

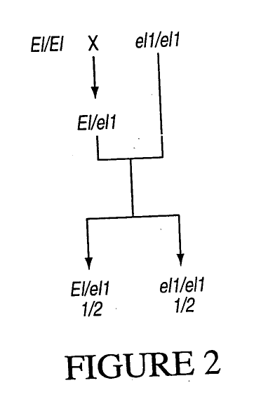

FIG. 2: The construction of a mapping population for the mutation el1 (elongate),

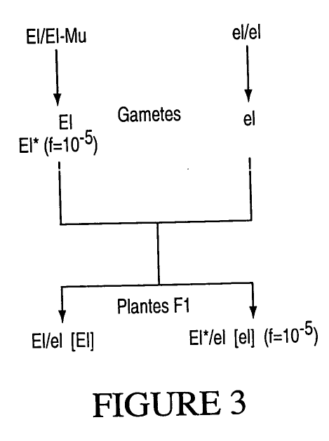

FIG. 3: The construction of a mutagenesis population for marking the elongate locus,

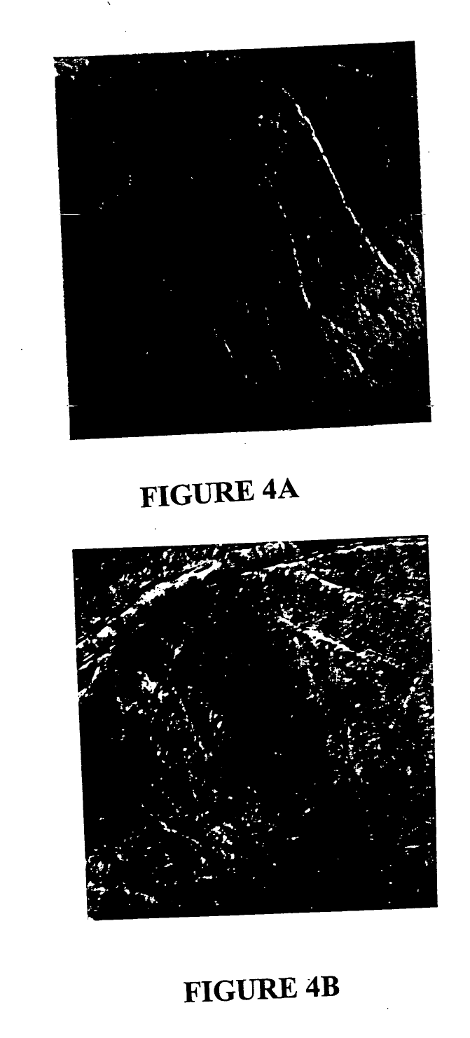

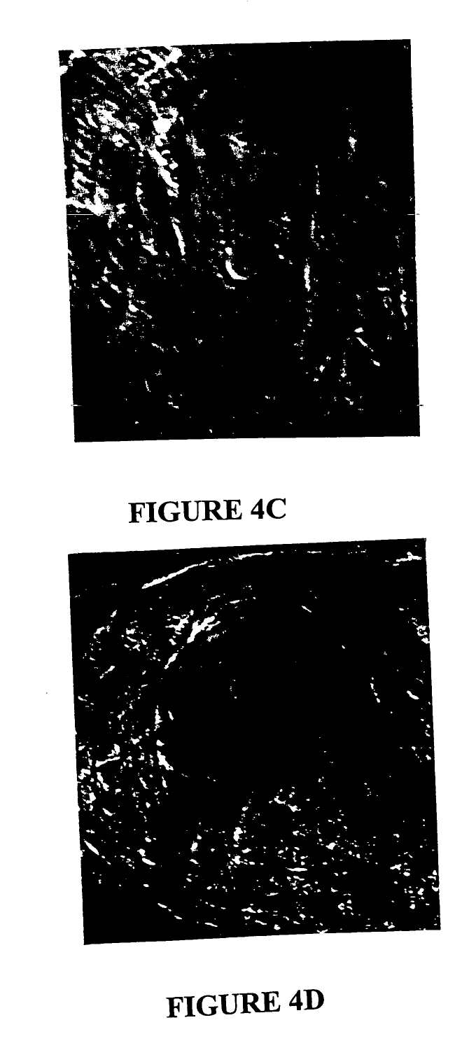

FIG. 4: The phenotypical characterization of megasporocytes in homozygous plants for the allele el1 and maize plants having the wild allele at the elongate locus, and the comparison with the sexed and apomictic forms in Tripsacum,

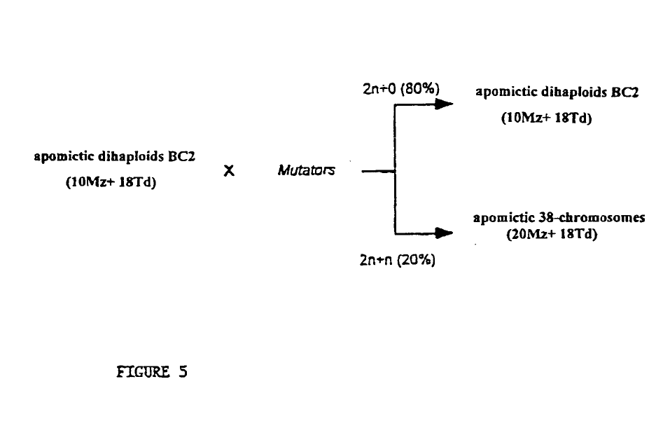

FIG. 5: The construction of a mutagenesis population in maize-Tripsacum hybrids, allowing functional analysis of the relationship between the sequence isolated in the apomictic plants and the expression of apomixis.

### EXAMPLE 1

### Genetic Mapping of Apomeiosis in Tripsacum

The production of a genetic map of the segment of the chromosome which controls apomeiosis in apomictic and sexed forms of the genus Tripsacum is described below.

1) Materials

Mapping of diploid Tripsacum: The two parents used are sexed diploid plants (2n=36) of the ORSTOM-CIMMYT collection, kept on the experimental station of Tlaltizapan, state of Morelos in Mexico. They are a Tripsacum maizar Hern. and Randolph, accession CIMMYT #99-1114, and a Tripsacum dactyloides var. meridionale de Wet and Timothy, accession CIMMYT #575-5136. The population comprises 175 F1 plants, among which 56 were used for the mapping.

Mapping of the apomeiosis: The mapping population comprises 232 F1 maize-Tripsacum plants. The parent maize (H1) is a hybrid of maize (2n=2x=20) between two CIMMYT lines (CML 135 by CML 139). The other is a tetraploid and apomictic Tripsacum dactyloides (2n=4x=72), accession CIMMYT #65-1234. The apomictic plant was used as the male.

2) Methods

Analysis of the modes of reproduction: This is carried out by the method of Leblanc et al. (4).

Detection of Molecular Markers of the RFLP Type Associated with Apomeiosis:

The strategy followed corresponds globally to that described by Michelmore et al. (14) for the detection of molecular markers associated with a specific phenotypical response.

The probes were obtained from the University of Missouri, Columbia.

About a hundred RFLP probes were chosen on the genetic maps which exist for maize, to obtain a density of about 20-30 cM between two markers (see (4), appendix 4). Various reference maps were used: the UMC map (University of Missouri, Columbia; Maize DataBase, map UMC95), a map of the University of Cornell (15) and various maps produced at CIMMYT (16). A chi2 test was used to detect potential links, and the recombination values were evaluated by the method of Allard (17). Since the donor parent of the segment which controls the apomeiosis is a heterozygous tetraploid plant, three conditions are necessary for detection of a link between diplospory and an RFLP allele: (1) existence of an RFLP polymorphism between the two parents at this locus, (2) heterozygosity for this allele in Tripsacum, (3) the allele must be simplex in the tetraploid, that is to say can be differentiated from the other 3.

Mapping of Diploids

An F1 mapping population between two heterozygous parents belonging to two distinct species was used. The mapping methods are as described by Ritter et al. (18).

3) Results

Identification of RFLP Markers Associated with Apomeiosis

FIG. 1 shows the genetic mapping of the chromosomal segment which controls apomeiosis, and comparison with the sexed diploid plants in Tripsacum and maize. “Apo” corresponds to the locus responsible for the apomeiosis. The position of umc71 on chromosome 6 of maize is indicated approximately, the allele on chromosome 6 having been withdrawn from the last versions of the UMC map. The map was developed from 52 plants of the F1 population between maize and Tripsacum. In this population, meiosis and diplospory are in 1:1 segregation (24 apomictic plants against 28 sexed plants, chi2=0.31, p=0.6). Eighty-four probes, selected on the UMC map to cover as broadly as possible the genome of maize, were tested. 90% of them detect at least a polymorphism between maize and Tripsacum. Three probes with a polymorphous allele specific to the apomictic bulk were tested on the total F1 population. One allele, detected by probe umc28, proved to be associated with apomeiosis. In a second stage, fourteen probes close to the locus of umc28 on the UMC map were tested. Four of them, umc71, umc62, csu68 and cdo202, detect RFLP alleles which are both associated with diplospory and in complete co-segregation between themselves.

Comparative Mapping Between Sexed Diploids and Apomictic Tetraploids.

The five markers associated with diplospory were mapped on the diploid population. All of them could be on the same parent (575-5136). It is noted that the five markers are all strictly linked in the tetraploid plant, but are separated by significant recombination values both in the diploid parent and in maize.

Comparative Mapping of Maize-Tripsacum:

Probes which detect alleles associated with the chromosomal segment which controls diplospory in Tripsacum detect all the alleles belonging to the same linkage group on the map of maize. This is the long arm of chromosome 6. Some of them also detect alleles in other regions of the genome, in particular chromosomes 3 and 8. The locations on the maize map of the various probes associated with apomeiosis are shown in the following table:

| Clones | Location |
|---|---|
| UMC38* | 6L; 8L; 3L; |
| UMC62 | 6L |
| UMC71 | 6L; 8L; |
| UMC28 | 6L |
| CSU68 | 6L; 8L; 3L |
| CDO202 | 6L; 8L; 3L |

*not associated with diplospory, but belong to the same linkage group.

### Example 2

### Identification of Orthologous Genes in Maize

The genes which lead to expression of apomeiosis in Tripsacum were investigated in maize. The aim was thus to indentify in maize candidate genes having both (1) the same genomic location as apomeiosis and (2) a phenotype related to apomeiosis.

There are very many mutants of meiosis in maize. The phenotypical criteria adopted in the choice of the candidate genes were the following: (1) the presence of clearly differentiated archesporial cells, (2) the total absence of induction of meiosis in these cells, or the failure thereof at an early stage, (3) the capacity to produce a functional gametophyte independently of the failure of the meiosis, (4) the absence of or at least a very marked drop in the depots of callose around the parent cell of the megaspore. Among the potential candidates, that is to say those having all or some of the abovementioned characteristics, those of which the position in the genome of maize was unknown or imprecise were mapped using as reference loci those detected by the probes used for mapping apomeiosis in Tripsacum.

Materials and Methods

The works of which the results are reported below were carried out on elongate (el1), which is a mutant of recessive meiosis (19). In plants which are homozygous for the el1 locus, the chromosomes remain decondensed during the metaphase and the anaphase of the first division, causing various chromosomal anomalies, of which a significant proportion are non-reduced gametophytes (30 to 70%, depending, inter alia, on the genetic base in which the mutation is placed). Fertilization by the pollen of a normal plant leads to a triploid embryo, and to a deficient pentaploid albumen.

The precise location of the elongate locus was unknown until the invention. However, it was known that it belonged to the long arm of chromosome 8 of maize. It was thus not located directly on the arm of the chromosome of maize identified by Leblanc et al. as homeologous to that which controls apomeiosis. Located on chromosome 8, however, it potentially belongs to a segment which is duplicated between the distal part of the long arm of chromosome 6 and certain parts of chromosome 8 (15).

FIG. 2 describes the construction of a mapping population for the mutation el1 (elongate). Three F1 plants of the heterozygous genotype El1/el1 were retro-crossed over three homozygous el1/el1 plants. For each family, 50 plants were grown, autofertilized and evaluated for their phenotype. The El1/el1 seeds are expected normal, while the el1/el1 seeds have a malformed albumen. In order to confirm the elongate phenotypes, ten to twenty embryos inside the seeds with a deficient albumen were sampled and analysed by flow cytometry using the protocol proposed by Galbraith et al. (20). The el1 mutation was obtained from the Maize Genetic Stock Center, Urbana, Ill., in the form of seeds resulting from autofertilization of plants homozygous for the el1 allele in a genetic base, which is furthermore indeterminate. The homozygous line of the wild phenotype, W23, was used for construction of the population. Detection of the linkage and estimation of the recombination values were achieved with the aid of Mapmaker 2.0 software for Mackintosh.

Comparison of the Phenotypical Expression of Apomeiotic Plants and Elongate.

Phenotypical expression of the elongate mutation in the archesporial cells of homozygous el1/el1 plants was analysed by cytoembryology techniques previously described by Leblanc et al. (11). Immature inflorescences were harvested on four types of materials: (1) sexed diploid Tripsacum (2) apomictic tetraploid Tripsacum, accessions described in Leblanc et al. (11), (3) a line of homozygous El1/El1 maize of the wild phenotype (W23), and (4) a homozygous el1/el1 line.

Tagging and Isolation of the Elongate Locus Sequence with the Aid of Transposable Elements.

Tagging by transposon consists of using transposons of which the sequence is known in order to create a mutation for a gene of which only the phenotypical expression is known. The insertion of the transposon in the gene which is to be isolated is often characterized by the loss of its function. In the case of recessive alleles, it is frequently characterized by the appearance of the recessive phenotype in heterozygous plants. The mutated gene can then itself be cloned by marking the site of insertion of the transposon. The mutated gene can thus itself be isolated by marking the site of insertion of the transposon, the various loci where transposons have been inserted being cloned by the conventional techniques of Mendelian analysis and molecular biology. The experiments below were carried out with the Mutator system (21).

The population used for tagging the elongate locus with the aid of transposons is shown as a diagram on FIG. 3 (f: frequency of appearance; [EL] and [el]: dominant and recessive phenotypes; El*: marked allele. Plants homozygous for the recessive mutation are crossed with plants homozygous for the wild allele, El1. In the population of gametes produced by the parent El1/El1, one or more mutations are found at the elongate locus. While the insertion leads to the loss of the function of this allele, some F1 plants of the el1/El1 genotype but el1 phenotype are thus found. The gene is thus marked.

Results:

Mapping of the Elongate Locus:

In the populations in segregation, the el1 and El1 phenotypes segregate in proportions of 1:1 (Chi2=0.5; p=0.6). Various RFLP probes belonging to chromosomes 3, 6 and in the majority of cases 8 were tested with three restriction enzymes EcoRI, BamHI, and HindIII on 50 plants of the population. Probes which detect polymorphisms of interest were then analysed on 100 supplementary individuals. Umc28, umc62 and umc71 show no polymorphous alleles associated with elongate with the three enzymes tested. Csu68 and cdo202, on the other hand, detect alleles associated with the elongate locus. The linkages between the three loci, estimated in recombination percentage, are as follows:

| | cdo 202 | csu68 |
|---|---|---|
| elongate | 9.3 | 7.4 |
| csu68 | 1.9 | |

Phenotypical Characterization:

FIG. 4 shows a comparison of the developments of archesporial cells in these various types of materials (A: parent cell of the megaspore in W23, a maize of genotype El1/El1; B: parent cell of the megaspore in a homozygous el1/el1 maize; C: parent cell of the megaspore in a sexed diploid Tripsacum; D: parent cell of the megaspore in an apomictic tetraploid Tripsacum). The development stages observed were identified and synchronized between the various forms observed on the basis of the size and morphology of the external teguments of the ovule (11). For the sexed forms in maize and Tripsacum: entirely similar development characteristics are observed: same morphology both of the cells and of the nuclei (inter alia: parent cell of the megaspore of rectangular shape, thick pronounced walls, very pronounced depots of callose at the cell walls, from the parent cell of the megaspores to the formation of the megaspore). This same similarity is observed on comparing the diplosporic forms in Tripsacum with homozygous El1/El1 maize plants: same morphology both of the nuclei and of the cells (direct development of the parent cell of the megaspore in the embryonal sac, fine cellular wall, nuclei very clearly different from those observed in the sexed forms, and very comparable between the diplosporic forms in Tripsacum and the el1/el1 plants in maize, and absence or very small depots of callose in the parent cell of the megaspore).

Tagging by Transposon of the Elongate Locus:

A population of 12,500 F1 plants produced according to FIG. 3 were grown on the experimental station of CIMMYT at Tlatizapan, Mexico. The 12,500 plants were fertilized by a maize hybrid deprived of active Mutator (hybrid CML135*CML 62). The ears of the 12,500 were monitored to maturity in order to detect those which express the elongate mutation although heterozygous at this locus. For the plants with deficient albumens, the corresponding embryos were analysed by flow cytometry. Two plants identified as TTEl-5 and TTEl-7 were identified as having deficient albumens associated with a triploid embryo. These plants express the elongate phenotype in heterozygous el1/El1 plants. In these plants, the wild allele at the locus was tagged by one of the transposons of the Mutator type. The seeds produced by crossing these two plants with the hybrid CML135*CML62 form a population in which this marked allele segregates at the elongate locus: half of the plants resulting from this crossing are of the el1/El genotype (el1 resulting from the TTEl plant, El resulting from CML135*CML62), the other half being the El1*/El genotype, where El1* is the allele marked by the transposon and resulting from the TTEl plants. The gene at the elongate locus can thus be identified and cloned by analysing the co-segregation of various copies of transposable elements present in these plants and of the elongate locus located with the RFLP probes mentioned subsequently.

### Example 3

### Production and Process for the use of a Mutagenesis Population to Confirm the Relationship Between the Candidate Allele and the Phenotypical Expression of All or Part of the Apomictic Development

The general plan and the materials used are shown on FIG. 5. The lines containing the Mutator elements were obtained from M. Freeling, University of California in Berkeley. The dihaploid BC2-28 plants used here are those described previously by Leblanc et al. (6). They are apomictic plants which have a haploid genome of each of the two parents of maize and Tripsacum origin. We used these plants to introduce Mutator elements into an apomictic material.

A population of a thousand apomictic BC2-28 plants was first constituted from a single apomictic dihaploid plant, and by selection of the plants of the 2n+0 type among its descendants. We thus create a thousand copies of the same apomictic polyhaploid genotype. These thousand plants were crossed with Mutator stocks. In the descendants obtained, we selected the out-of-type 2n+n plants, that is to say those which have incorporated a genome resulting from the Mutator stocks. These stocks have about 200 copies of various types of Mutators. It is thus hoped to recover on average 100 copies in the apomictic plants. Selection of the BC2-28 plants and the out-of-type apomictic BC3-38 plants is made on a simple morphological criterion. The dihaploids in fact have a very recognizable phenotype which is very different from that which results from accumulation of a supplementary maize genome (6). In total, about 35,000 seeds (BC2-28×Mutators) were obtained. All were grown on the experimental station of CIMMYT at Tlaltizapan, state of Morelos, in the course of the summer of 1996. The out-of-type 2n+n were selected one month after germination. About 7,500 2n+n plants were obtained, that is to say almost 20% of the out-of-type plants. This population was recrossed with a maize hybrid (CML135*CML62) deprived of active Mutator elements, producing a population of about 150,000 seeds, representing the inverse genetic population.

This population represents ideal material for analysing the effect of a given sequence on the expression of apomixis. In fact, the Tripsacum alleles responsible for expression of this characteristic are in a simplex condition here (a single copy in the genome). To verify the allele-function relationship, it is thus sufficient to check the phenotypical effect of the insertion of a transposon in this sequence. If the mutation induces a loss of function, the sequence-function relationship has thus been established with certainty. Since the sequences both of the transposons and of the gene studied are known, plants which have been mutated for this allele can be identified by the conventional PCR techniques.

### Example 4

### Mapping of Insertions and Cloning of the Flanking Regions.

Locus Cdo 202 which was mapped close to Elongate locus was used as reference for identifying the insertions of interest.

A method derived from the AFLP technology (EP 0 534 858) was used to isolate the DNA fragments co-segregating with Cdo 202. The insertion sites of the transposons and their segregation into a population were thus visualized on a gel (the detection of the transposon insertions are given at the end of the Example).

A population segregating for the tagged and wild el1 allele was used.

The segregation of the allele detected by Cdo 202 on said population was compared to those of the different insertion sites of the transposons.

The insertions are thus mapped with reference to the allele of Cdo202, which is located on the mapped population at about 7 cM of Elongate.

The fragments located between 0 and 15 cM of allele Cdo202 were cloned (the bands were exised of the Nylon^{R} membrane, washed twice with 500 μl H₂O and the DNA eluted by incubating into 30 μl H₂O, 92° C., 10 min. 2 μl of the elution product were used for re-amplifying the band in the same PCR conditions and with the same primers.

The amplified fragments were cloned in pGEM-T, according to the manufacturer's recommendations (Promega).

The inserts-containing pGEM-T vectors were introduced into E. coli (XL-Blue) and the transformed colonies directly screened by PCR with universal primers (CV 72 and CV 76). The inserts were then sequenced on an automatic sequencer ABI 377, according to the protocol given by Perkin-Elmer.

Based on their position on the map, 3 fragments were isolated, cloned and sequenced.

SEQ ID N° 1, SEQ ID N° 2 and SEQ ID N° 3 correspond to the sequences of the fragments after substraction of the bases corresponding to the adaptators Mse, the vector and the transposon fragment.

SEQ ID N° 1 clone 54-9
GAAGAGGTAAAATAGAGTTTCGATTTGGT
SEQ ID N° 2 clone 56-7
CTCGACNNCAAACCCTAATCGACACTTTGAGAGGANNGGATCCCCTAGG
SEQ ID N° 3 clone 57-18
ACAACTACACTAACTGAGCCCAGCCCAATCCAAGCCTATGCCGCTCGAC-
GCTCGTTCTCACTTTCTCAGCCGAGA

Homologies were observed with two fragments within public databases: Histone H1-1 from Arabidopsis thaliana, and Cdc36 (NOT2) (a yeast gene involved in the control of the cell cycle).

Regarding H-1, the isolated fragment is located at the junction between an intron and an exon, for histone H1-1, and the transposon insertion is within the exon.

With regard to Cdc36, the fragment is part of an exon.

The homologies between SEQ ID N° 1 and H1-1 (SEQ ID N° 4) on the one hand and SEQ ID N° 1 and Cdc36 (SEQ ID N° 5) on the other hand, are as follows:

SEQ ID N° 1

| | | |
|---|---|---|
| 1 | GAAGAGGTAAAATAGAGTTTCGATTTGGT | 29 |

SEQ ID N° 4

| | | |
|---|---|---|
| 1530 | GAAGAGGTAAAAATGAGATTCGATTTCGT | 1550 |
| | ************ *** ******** ** | |

SEQ ID N° 1

| | | |
|---|---|---|
| 1 | GAAGAGGTAAAATAGAGTTTCGATTTGGT | 29 |

SEQ ID N° 5

| | | |
|---|---|---|
| 3301 | GAAGAGGAAAAATAGAGTTTCATCTTGAAA | 3350 |
| | ******* ************ *** | |

Detection of Mutator's Insertions.

300 ng DNA were digested 2 h, 37° C., with 1 U Ecor I (Life Technologies), total volume of 20 μl, following the protocol given by the manufacturer.

Adaptators Mse (final concentration: 1 μM), such as disclosed in EP 0 534 858, were ligated to 75 ng of restricted DNA (2 h, at the ambient, in the presence of 0.2 U of T4 ligase (Life Technologies), with a buffer such as recommended by the manufacturer. The reaction was diluted 10 times in H₂O. 2 μl of said dilution were used in a PCR reaction with a primer conjugated to digoxygenine having SEQ ID N° 6,

CCCTGAGCTCTTCGTCYATAATGGCAATTATCTC

wherein Y represents A or T, (0.5 μM final), Mse-N primer, wherein N is C, A, T or G, 200 mM dNTP, 1.5 mM MgCl₂, Taq polymerase, and a buffer.

The PCR profile consists of 35 cycles, each cycle corresponding to 94° C., 30 sec; 58° C., 60 sec., 72° C., 60 s.

3 μl of the amplification products are migrated on a gel under denaturating conditions. After the electrophoresis, the DNA was transferred by capillarity on a Nylon^{R} membrane (Biodyne A^{R}, Life Technologies), and the digoxygenine-labelled fragments were first revealed by chemioluminescence, then by colorimetry in the presence of BCIP and NBT, according to the manufacturer's protocol (Life Biotechnologies).

Bibliography

(1) Savidan, Y., 1982 Nature et hérédité de l'apomixie chez Panicum maximum Jacq. Universite of Paris XI.

(2) Nogler, G. A., 1984a Genetics of apospory in apomictic Ranunculus auricomus V. Conclusions. Bot Helvetica 94: 411-422.

(3) Mogie, M., 1988 A model for the evolution and control of generative apomixis. Biol J Linnean Soc 35: 127-153.

(4) Leblanc, O., M. D. Peel, J. G. Carman and Y. Savidan, 1995a Megasporogenesis and megagametogensis in several Tripsacum species (Poaceae). Am J Bot 82: 57-63.

(5) Hanna, W., M. Dujardin, P. Ozias-Akins, E. Lubbers and L. Arthur, 1993 Reproduction, cytology, and fertility of pearl Millet X Pennisetum squamulatum BC4 plants. J Hered 84: 213-216.

(6) Leblanc, O., D. Grimanelli, N. Islam-Faridi, J. Berthaud and Y. Savidan, 1996 Reproductive behaviour in maize-Tripsacum polyhaploid plants; implications for the transfer of apomixis into maize. J Hered 87: 108-111.

(7) Kindiger, B., V. Sokolov and I. V. Khatypova, 1996 Evaluation of apomictic reproduction in a set of 39 chromosome maize-Tripsacum backcross hybrids. Crop sci 36: 1108-.

(8) Jefferson, R. A., and R. Bicknell, 1996 The potential impact of apomixis: a molecular genetic approach In The impact of plant molecular genetics, edited by B. W. S. Sobral. Birkhaüser, Boston.

(9) Koltunow, A. M., R. A. Bicknel and A. M. Chaudhury, 1995 Apomixis: molecular strategies for the generation of genetically identical seeds without fertilization. Plant Physiol 108: 1345-1352.

(10) Ramulu, K. S., P. Dijkhuis, A. Pereira, G. C. Angenent, M. M. Van Lookeren Campagne and J. J. M. Dons, 1997 EMS and transposon mutagenesis for the isolation of apomictic mutants in plants. In Somaclonal variation and induced mutations in crop improvement. Kluwers academic Publishers, Amsterdam.

(11) Leblanc, O., D. Grimanelli, D. Gonzaléz de Léon and Y. Savidan, 1995b Detection of the apomictic mode of reproduction in maize-Tripsacum hybrids using maize-RFLP markers. Theor Appl Genet 90: 1198-1203.

(12) Walbot, V., 1992 Strategies for mutagenesis and gene cloning using transposon tagging and T-DNA mutagenesis. Ann Rev Plant Physiol Plant Mol Biol 43: 49-82.

(13) Freeling, M. and Walbot, V. (eds), 1994 The Maize Handbook, Springer Verlag, New York.

(14) Michelmore, R. W., I. Paran and R. V. Kesseli, 1991 Identification of markers linked to disease-resistance genes by bulked segregant analysis: A rapid method to detect markers in specific genome regions by using segregating populations. Proc Natl Acad Sci USA. 88: 9828-9832.

(15) Ahn, S. N., and S. D. Tanksley, 1993 Comparative linkage maps of the rice and maize genomes. Proc Natl Acad Sci USA 90: 7980-7984.

(16) Ribaut, J. M., D. A. Hoisington, J. A. Deutsch, C. Jiang and D. Gonzalez-de-Leon, 1996 Identification of quantitative trait loci under drought conditions in tropical maize. 1. Flowering parameters and the anthesis-silking interval. Theor Appl Genet 92: 905-914.

(17) Allard, R. W., 1956 Formulas and tables to facilitate the calculation of recombinaison values in heredity. Hilgardia 24: 235-278.

(18) Ritter, E., C. Gebhardt and F. Salamini, 1990 Estimation of recombination frequencies and construction of RFLP linkage maps in plants from crosses between heterozygous parents. Genetics 125: 645-654.

(19) Rhoades, M. M., and E. Dempsey, 1966 Induction of chromosome doubling at meiosis by the elongate gene on maize. Genetics 54: 505-522.

(20) Galbraith, D. W., K. R. Harkins, J. M. Maddox. N. M. Ayres, D. P. Sharma and E. Firoozabady, 1983 Rapid flow cytometric analysis of the cell cycle in intact plant tissues. Science 220: 1049-1051.

(21) Robertson, D. S., 1980 The timing of Mu activity in maize. Genetics 94: 969-978.

1/ Process for identifying in a Gramineae, and more particularly in a maize, a nucleotide sequence orthologous to the sequence responsible for all or some of the apomictic development in an apomictic form, characterized in that mutations having a phenotype close or similar to that observed in an apomictic form are mapped in the genome of the Gramineae, more particularly that of a maize, to identify those which appear orthologous to genes involved in apomixis.

2/ Process according to claim 1, characterized in that meiotic mutations are mapped in the genome of the Gramineae, more particularly of a maize, to identify those which appear orthologous to genes involved in apomixis.

3/ Process according to claim 2, characterized in that the position of the various meiotic mutations in the genome of the Gramineae, more particularly of a maize, is located with the aid of molecular markers capable of locating the loci responsible for apomeiosis in the said apomictic form.

4/ Process according to claim 3, characterized in that molecular markers which are capable of locating the loci responsible for diplospory in Tripsacum are used.

5/ Process according to claim 4, characterized in that the said location relates to the elongate and afd loci.

6/ Process according to any one of claims 1 to 5, characterized in that it also comprises tagging the meiotic mutations located, by a transposon.

7/ Process according to claim 6, characterized in that the tagging by a transposon is carried out at the elongate locus with the aid of transposable elements of the Mutator or Ac/Ds type.

8/ Process according to any one of the above claims, characterized by the cloning and sequencing of the mutations located.

9/ Process according to claim 6 or 7, characterized in that the mutated genes are cloned, after the site of insertion of the transposon has been marked by segregation analysis, and in that they are sequenced, if desired.

10/ Nucleotide sequences, characterized in that they are orthologous to the sequences responsible for all or some of the apomictic development in an apomictic form and the homologous sequences.

11/ Nucleotide sequence according to claim 10, characterized in that it corresponds to a mutated elongate gene.

12/ Nucleic acids containing one or more sequences as defined in claim 10 or 11, associated with the regulatory sequences necessary for expression in a plant material.

13/ Cloning and expression vectors containing nucleic acids according to claim 12.

14/ Cell hosts containing a vector according to claim 13.

15/ Use of a sequence according to claim 10 or 11, if appropriate in conjunction with other alleles characteristic of apomictic forms, for introduction into the genome of a plant material, plant cells, plants at various stages of development and seeds, in order to impart to them an apomictic development.

16/ Plant cell of Gramineae, in particular of maize, characterized in that it contains in its genome at least the part of a sequence according to claim 10 or 11 involved in an apomictic development.

17/ Plant of the family of Gramineae, in particular maize, characterized in that it contains in its genome at least the part of a sequence according to claim 10 or 11 involved in an apomictic development.

18/ Seed of Gramineae, in particular maize, characterized in that it contains in its genome at least part of a sequence according to claim 10 or 11 involved in an apomictic development.

19/ Process for the production of apomictic plants, characterized in that a nucleotide sequence according to claim 11 is used.

20/ Use of at least a part of a sequence according to claim 10 or 11 for identifying and isolating the orthologous sequences of loci in apomictic forms.

21/ Hybridization probes and molecular primers, characterized in that they are compiled from a sequence according to claim 10 or 11.

22/ Hybridization probes and molecular primers according to claim 18, characterized in that they are compiled from the elongate sequence.

23/ Process for identifying and isolating genes responsible for apomeiosis in apomictic Tripsacum, characterized in that at least a part of the sequence of the elongate locus is used.

24/ Process for the use of a mutagenesis population to confirm the relationship between a sequence isolated in Tripsacum according to claim 20 and expression of apomixis.
